(19) United States
(12) Patent Application Publication
(10) Pub. No.: US 2007/0048243 A1
(43) Pub. Date: Mar. 1, 2007

(54) Anti-aging composition containing criste marine and padina pavonica extracts

(75) Inventors: Isabelle Hansenne, Westfield, NJ (US); Hani Fares, Somerset, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 11/215,691

(22) Filed: Aug. 30, 2005

(51) Int. Cl. A61K 8/97; A61K 36/02; A61K 36/18

(52) U.S. Cl. 424/74; 424/195.17; 424/725

(57) ABSTRACT

The present invention relates to topical compositions that include both Criste Marine and Padina Pavonica and their use for improving various properties of skin, including its appearance.

### BACKGROUND OF THE INVENTION

In the aging process, various signs appear on the skin resulting from a modification of skin structure and of cutaneous function. One of the clinical signs of aging are, among others, the appearance of fine wrinkles and deep wrinkles. These signs of aging increase with age. A disorganization of skin “texture” is more particularly observed, meaning that micro-contours are less regular and show an anisotropic character.

It is known to treat these signs of aging using cosmetic or dermatological compositions containing actives adapted to fight against such signs of aging, such as alpha-hydroxy-acids, beta-hydroxy-acids and retinoids. Such actives act on wrinkles primarily by eliminating dead skin cells and accelerating the cell renewal process.

Several compositions have been suggested which have a tensing effect, making it possible to obtain an immediate skin smoothing. Such compositions generally contain as a tensing agent substances from natural origin, such as plant, egg, milk or animal derivatives. For instance, the International Application WO-98/29091 discloses the use, as a tensing agent, of a polymeric system having particular physico-chemical properties and comprising at least one polymer from natural origin, such as a soya protein extract or chitin or keratin derivatives. Moreover, the International Application WO 96/19180 discloses toning compositions comprising a film-forming agent containing at least one plant polysaccharide and hydrolyzed casein as tensing actives. However, the use of substances from natural origin is limited by hazards associated with bovine spongiform encephalopathy. Moreover, not only are tensing effects not always reproducible, but the tensing effect obtained from such substances is not very significant from a qualitative standpoint, and from a quantitative standpoint, the effect shows a low remanence.

Other prior art compositions having a tensing effect use synthetic polymers. PCT Application WO-98/29022 discloses a composition having a tensing effect and comprising an aqueous dispersion of a polymeric system containing at least one polymer from plastic origin selected from the group comprising several types of polyurethanes, polyureas, acrylic polymers or copolymers, sulfonated isophthalic acid polymers and the mixtures thereof. The cosmetic feel of such compositions is, however, not satisfactory. Additionally, European Patent Application EP-1038519-A1 discloses the use as a tensing agent in and/or for the manufacture of an anti-wrinkle composition, of at least one grafted silicone polymer comprising one polysiloxane portion and one portion consisting of a non silicone organic chain, one of the two portions constituting the main chain and the other one being grafted on said main chain. However, such silicone polymers do not always exhibit satisfactory anti-wrinkle effects when they are formulated in an emulsion.

The use of mineral fillers and more particularly of silica in cosmetic compositions is known. For instance, silica is a widely used material in the cosmetic industry. It confers softness and a matte aspect to the formulations. It is also used in association with titanium dioxide or zinc oxide for forming complex particles with sun filter effects. For such applications, silica is very often in pulverulent form and a wide range of particle sizes is available from 1 μm to 100 μm. However it is far less usual, and even exceptional, to use colloid particles.

European Patent Application EP-1 008 334-A2 discloses the use and preparation, at an acidic pH, of mixed silicates, as tensing agents, for tensing and smoothing the skin, in order to immediately attenuate the skin wrinkles and/or fine wrinkles, the mixed silicates being in the form of particles having an average size ranging from 15 to 1000 nm resulting in colloid solutions. However, nothing is mentioned regarding fillers other than mixed silicates. Moreover, the tensing effect and the smoothing power are attributed to the chemical nature of the mixed silicates and not to the particle size.

Retinol (vitamin A) is an endogenous compound that occurs naturally in the human body and is essential for normal epithelial cell differentiation. Natural and synthetic vitamin A derivatives (retinoids) have been used extensively in the treatment of a variety of skin disorders and have been used as skin repair or renewal agents. Retinoic acid, for example, has been employed to treat a variety of skin conditions, e.g., acne, wrinkles, psoriasis, age spots and discoloration. See, e.g., Vahlquist, A. et al., J. Invest. Dermatol., Vol. 94, Holland D. B. and Cunliffe, W. J. (1990), pp. 496-498; Ellis, C. N. et al., “Pharmacology of Retinols in Skin”, Vasel, Karger, Vol. 3, (1989), pp. 249252; Lowe, N. J. et al., “Pharmacology of Retinols in Skin”, Vol. 3, (1989), pp. 240-248, PCT Patent Application No. WO 93/19743. Compounds which can act similarly on skin may be referred to as “retinoid-like” or as providing “retinoid-like” activity or function.

In addition to alpha-hydroxy-acids, beta-hydroxy-acids and retinoids, a number of active species have been described for use in anti-aging products based on vitamins such as ascorbic acid which can influence wrinkling by effecting the junction between various layers and structures within the skin. Ascorbic acid (or vitamin C). For example, vitamin C has been described for use in topical administration to reinforce the cohesion of the dermo-epidermal junction which can contribute to firmer, more elastic skin. See Bernerd, U.S. patent application publication No. 2004/0005342 A1.

In cases where the anchoring structures are impaired (deficiency of collagen VII or tenascin synthesis, aging, etc.), this causes flattening of the dermo-epidermal junction. The exchanges are reduced, the two tissues are less solidly attached, the epidermis folds and, since the skin is less firm and less taut, wrinkles appear and the skin becomes more susceptible to mechanical attack. This can be addressed by the use of ascorbic acid. See id. See also U.S. Pat. Nos. 6,908,925; 6,896,889; 6,231,877; PCT published application nos. WO 02/056856 and WO 0108649; and FR-2,657,011.

Thus while there are a number of known an speculated causes and solutions to skin aging, and in particular, the occurrence of fine lines and wrinkles, there remains a need, for the discovery of new and useful materials that can act to decrease the appearance of fine lines and wrinkles, assist in their long-term treatment, or both. There is also a need to find safe, natural and renewable materials to provide these benefits.

### SUMMARY OF THE INVENTION

In another aspect, the present invention includes cosmetic and dermatological topical compositions that contain extracts from marine plants (which includes plants that grow near oceans and seas, as well as those that grow in oceans and seas) that are useful in providing at least one of improvement in firmness/elasticity of skin to which it is applied or softening the look of lines and wrinkles thereof, particularly on the face and/or hands, wherein the extracts from marine plants exhibit at least one of retinol-like properties, antioxidant properties, tensioning properties, cell renewal enhancing properties or hydrating properties and wherein the marine plant extracts include both an extract from Criste Marine and an extract from Padina Pavonica.

In another aspect, there is provided a cosmetic and/or dermatological, topical compositions that consists essentially of extracts from marine plants and in particular, both an extract from Criste Marine and an extract from Padina Pavonica.

Methods of making such formulation and methods of their use are also included. In particular, in one aspect, the present invention provides a method of improving the firmness and/or elasticity of skin and/or visibly softening the look of lines and wrinkles of skin comprising the steps of application to skin in need thereof of a formula or composition that contain extracts from marine plants and, in particular, both an extract from Criste Marine and an extract from Padina Pavonica, at least once a day for and repeating the application for a time sufficient to provide either improved firmness, improved elasticity or visibly softened look to lines and wrinkles of the skin to which it is applied. In a preferred embodiment, the skin so treated is on the face or hands. This can be measured by standard techniques such as by biomechanical studies on human volunteers using a Torque Meter. (See Example IV.)

In one embodiment in accordance with the present invention, cosmetic and/or dermatological topical compositions are described which include both Criste Marine and Padina Pavonica and make it possible to obtain advantages in accordance with the present invention as described herein (such as, without limitation, improvements in firmness, elasticity, or softening of the appearance of fine lines and wrinkles of the skin to which it is applied) to a level not obtainable through the use of either Criste Marine or Padina Pavonica alone in an equivalent amount.

In another embodiment of the present invention, the cosmetic and/or dermatological topical compositions of the present invention containing both Criste Marine and Padina Pavonica provide statistically significant increases in, for example, elasticity and firmness over controls as well as improvement when comparing to like formulations without one or the other of these two active ingredients.

In yet another embodiment, there is provided a cosmetic and/or dermatological topical composition comprising at least about 0.025% by weight of the final formulation of an extract of Criste Marine and at least about 0.025% by weight of the final formulation of an extract of Padina Pavonica. Preferably, the weight of each extract ranges from about 0.025% to about 10% by weight of each ingredient relative to the weight of the final composition in yet another embodiment between about 0.025% and about 5% by weight of each. The extract of Criste Marine in accordance with the present invention will preferably have at least 350 milligrams per kilogram of phytosterols or at least about 100 milligrams per kilogram of total tocopherols. The Padina Pavonica use should exhibit a biological activity of at least about 100,000 U Tex/Liter.

### DETAILED DESCRIPTION

While the specification concludes with the claims particularly pointing and distinctly claiming the invention, it is believed that the present invention will be better understood from the following description. All percentages and ratios used herein are by weight of the total composition and all measurements made are at 25° C. and normal pressure unless otherwise designated. All temperatures are in Degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Preferably, such additives will not be present at all or only in trace amounts. However, it may be possible to include up to about 10% by weight of materials that could materially alter the basic and novel characteristics of the invention as long as the utility (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

Criste Marine, pronounced KRISS-teh muh-REEN, belongs to the Latin genus ‘crithmum maritimum’ and is an extract with antioxidant and/or retinol-like properties taken from a type of plant commonly known as rock, golden or marsh samphire. This mineral-rich, edible algae grows on rock formations along the Mediterranean Sea and sustains itself by absorbing nutrients from sea mist and high waves. (Note that Criste Marine may be spelled in a number of different ways including, without limitation, Cryste Marine.)

Criste Marine used in accordance with the present invention is an extract that includes a number of potentially active species. In particular, Criste Marine extract preferably contains at least about 350 milligrams per kilogram of phytosterols or at least about 100 milligrams per kilograms of tocopherols based on assays for the total of these classes of compounds. In another embodiment of Criste Marine extract includes at least about 700 milligrams/Kg or at least about 200 milligrams/Kg of phytosterols and tocopherols respectively. It will be appreciated that this is an indication of possible activity and the amounts of phytosterols and tocopherols is not limited to those ranges. Indeed, the only true upper limit for each is one of cost and practicality.

The lower limits may also be determined for a given formulation based on performance such as providing a statistically significant improvement in one of the recited advantages herein. Generally, however, the amount of Criste Marine used will be at least about 0.025% by weight and in another embodiment, at least about 0.05% by weight. The amounts are based on the weight of the final composition or formulation. The amount can range from 0.025% to about 10.0% and, in another embodiment 0.025% to about 5.0% by weight of the final composition. Criste Marine which may be used in accordance with the invention includes, without limitation, that available from CODIF, 70 rue de Commandant 1′ Herminier-BP43, 35406 Saint Malo cedex France designated HUILE DE CRISTE/SAMPHIRA OIL which is an 5% solution of Criste Marine extract in solvent. The exact content, the concentration and the amount of Criste Marine used may vary from application to application so long as the resulting product provides the advantages described in accordance with the present invention. In one embodiment, the amount of Criste Marine supplied in any given product should be sufficient such that some statistically significant increase in one or more of the properties described herein is objectively measurable, such as by the techniques described in Example IV hereof.

Padina Pavonica, pronounced puh-DEEN-uh′puh-VAH-nik′-uh, is extracted from the protective coating of a brown algae found in the Mediterranean Sea. It is believed to help improve the firmness and elasticity of the skin. Padina Pavonica used in accordance with the present invention is an extract that includes a number of potentially active species. Padina Pavonica may be purchased from Alban Muller, designated HPS3 Padina Pavonica which is a 10% solution of Padina Pavonica extract in solvent. Alban Muller International is located at 8, rue charles Pathé-94300 Vincennes-France. The exact content, the concentration and the amount of Padina Pavonica used may vary from application to application as described above in connection with Criste Marine, namely, the ability to obtain a statistically significant improvement in a measure described herein compared to control as described in Example IV. Alternatively, the Padina Pavonica used should exhibit a biological activity of at least about 100,000 U Tex/Liter. In still another embodiment, the Padina Pavonica extract will have a biological activity of at least about 200,000 U Tex/Liter. Preferred delivery formats include creams, lotions, gels, gel-creams, sticks, and serums. Again, this may vary depending upon the concentration and activity of the extract.

The amount of Padina Pavonica present is an amount sufficient to provide improvement in the degree of firmness, elasticity or softening of the appearance of fine lines and wrinkles in skin to which it is applied in a clinically demonstrable way. It is also worthy of note that by the use of both Criste Marine and Padina Pavonica it is possible, in some embodiments, to obtain more of the advantages of the present invention to a level not obtainable through the use of either Criste Marine or Padina Pavonica alone in an equivalent amount. Thus, a formulation including 1% by weight of the formulation of Criste Marine and 1% of Padina Pavonica by weight of the formulation would provide advantages even over a formulation including 2% of Criste Marine alone or 2% of Padina Pavonica alone.

The amount of Criste Marine present is noted above and is controlled by the factors previously described. These same factors also control the amount of Padina Pavonica provided with one addition—the amount of Padina Pavonica can also be influenced by the amount of Criste Marine present. However, the amount of Padina Pavonica present generally ranges from as little as 0.025% by weight of the final composition or formulation. In another embodiment, it can be present in amount of as little as 0.025%. The amount of Padina Pavonica can range from 0.05% to about 10%, more preferably from about 0.025% to about 5 by weight. Again, this may vary depending upon concentration and/or activity. Note, in a particularly preferred embodiment the amount of Criste Marine is greater than or equal to the amount of Padina Pavonica provided in the formulation. The delivery formats described above as preferred for Criste Marine are also preferred for the combinations. In another embodiment, the amount of Padina Pavonica is present in an amount which is equal to or greater than the amount of Criste Marine.

Note also that the amounts of Criste Marine and Padina Pavonica described herein assume the minimum amounts of phytosterols/tocopherols and/or U Tex/Liter as described herein. The amounts may vary depending upon the assays of the activity of the extracts in a proportional manner. Thus, if a more dilute extract of Padina Pavonica, for example, were used, one having only 50,000 U Tex/Liter, or Criste Marine extract of only 50 mg/kg of tocopherols, then it might be necessary to use at least 0.05% by weight of the active. It is not important to use less if the activity of these actives is higher and/or more concentrated.

In a particularly preferred embodiment, there are provided topical cosmetic or dermatological formulations which include Criste Marine and Padina Pavonica as described above with additional active ingredients, and in particular ascorbic acid (Vitamin C) and its derivatives and analogs. Ascorbic acid in accordance with the present invention is preferably provided in the form of ascorbyl palmitate and/or magnesium ascorbyl phosphate. Both “Vitamin C” and “ascorbic acid” are used generally herein unless the context implies otherwise. The amount of Vitamin C provided in accordance with the present invention can be affected by the various conditions described previously for both Criste Marine and Padina Pavonica but, in addition, amounts of those two other marine-based actives can exert an influence on the amount of ascorbic acid or equivalent thereof provided.

Vitamin C which may be used includes, without limitation, ascorbic acid, ascorbyl palmitate and magnesium ascorbyl phosphate is often a white, crystalline vitamin found in citrus fruits, tomatoes and green, leafy vegetables. An important antioxidant, it also offers clarifying benefits. One of its stabilized, synthetically derived version, magnesium ascorbyl phosphate, is an equally effective antioxidant and helps to regulate the biosynthesis of collagen.

To give an order of magnitude to the amount used, an effective amount preferentially represents from 0.001% to 20% relative to the total weight of the composition, preferably from 0.1% to 15% relative to the total weight of the composition and advantageously from 1% to 10% relative to the total weight of the composition. On account of its chemical structure (a-keto lactone) which makes it very sensitive to certain environmental parameters such as light, heat and aqueous media, it may be advantageous to use the ascorbic acid in the form of a saccharide ester of ascorbic acid or a metal salt of phosphoryl ascorbic acid.

The saccharide esters of ascorbic acid that may be used in the invention are especially the glucosyl, mannosyl, fructosyl, fucosyl, galactosyl, N-acetylglucosamine and N-acetylmuramic derivatives of ascorbic acid, and mixtures thereof, and more especially ascorbyl-2 glucoside or 2-O-alpha-D-glucopyranosyl L-ascorbic acid or 6-O-beta-D-galactopyranosyl L-ascorbic acid. The latter compounds and processes for preparing them are described in particular in documents EP-A-487,404, EP-A-425,066 and J 05 213 736.

With regard to the metal salt of phosphoryl ascorbic acid, it may be chosen from alkali metal ascorbyl phosphates, alkaline-earth metal ascorbyl phosphates and transition metal ascorbyl phosphates. The ascorbic acid analogues are, more particularly, its salts, such as, especially, sodium ascorbate, magnesium or sodium ascorbyl phosphate, its esters, such as, especially, its acetic, propionic or palmitic esters, or its sugars, such as, especially, glycosyl ascorbic acid.

Other Possible Actives

Among the possible additional actives, actives in addition to those from the marine plants include anti-glycation agent.

The term “anti-glycation agent” means a compound for preventing and/or reducing the glycation of skin proteins, in particular of dermal proteins such as collagen. Examples of anti-glycation agents are plant extracts of the Ericacea family, such as an extract of blueberry (Vaccinium angustifolium); ergothioneine and its derivatives; and hydroxystilbenes and their derivatives, such as resveratrol and 3,3′,5,5′-tetrahydroxystil-bene. These anti-glycation agents are described in patent applications FR 99/16166, FR 00/08158, FR 99/09267 and FR 99/16168, respectively. Resveratrol is particularly preferred for use in this invention.

Among the other possible active agents which may be used along with the marine plant extracts are those useful for stimulating dermal macromolecules, mention may be made of those that act: either on collagen synthesis, such as extracts of Centella asiatica; asiaticosides and derivatives; synthetic peptides such as lamin, biopeptide CL or the palmitoyl oligopeptide sold by Sederma; peptides extracted from plants, such as the soybean hydrolysate sold by Coletica under the trademark Phytokine®; plant hormones such as auxins or on elastin synthesis, such as the extract of Saccharomyces cerivisiae sold by LSN under the trademark Cytovitin®; and the extract of the alga Macrocystis pyrifera sold by SECMA under the trademark Kelpadelie®; or on glycosaminoglycan synthesis, such as the product of fermentation of milk with Lactobacillus vulgaris, sold by Brooks under the trademark Biomin Yogourth®; and the extract of Saccharomyces cerevisiae available especially from the company Silab under the trademark Firmalift® or from the company LSN under the trademark Cytovitin.® or on fibronectin synthesis, such as the extract of the zooplankton Salina sold by Seporga under the trademark GP4G®; the yeast extract available especially from the company Alban Muller under the trademark Drieline®; and the palmitoyl pentapeptide sold by Sederma under the trademark Matrixil® or on metalloprotease (MMP) inhibition, such as, more particularly, MMP 1, 2, 3 or 9.

Other actives may also include retinoids and derivatives, isoflavonoids, oligopeptides and lipopeptides, lipoamino acids, the malt extract sold by Coletica under the trademark Collalift®; extracts of blueberry or of rosemary; lycopene; isoflavones, their derivatives or plant extracts containing them, in particular extracts of soybean (sold, for example, by Ichimaru Pharcos under the trademark Flavosterone SB®), of red clover, of flax, of kakkon or of sage; or on the inhibition of serine proteases such as leukocyte elastase or cathepsin G.

Other actives may also include the peptide extract of Leguminosa seeds (Pisum sativum) sold by LSN under the trademark Parelasty®; and heparinoids and pseudodipeptides. Among the active agents that stimulate epidermal macromolecules, such as fillagrin and keratins, mention may be made especially of the extract of lupin sold by Silab under the trademark Structurine®; the extract of beech Fagus sylvatica buds sold by Gattefosse under the trademark Gatuline®; and the extract of the zooplankton Salina sold by Seporga under the trademark GP4G®. The agents for stimulating the proliferation of fibroblasts that may be used in the composition according to the invention may be chosen, for example, from plant proteins or polypeptides, extracts, especially of soybean (for example an extract of soybean sold by LSN under the name Eleseryl SH-VEG 8® or sold by Silab under the trademark Raf fermine®); and plant hormones such as giberrellins and cytokinins. The agents for stimulating keratinocyte proliferation that may be used in the composition according to the invention especially comprise retinoids such as retinol and its esters, including retinyl palmitate; extracts of nut cakes sold by Gattefosse; and extracts of Solanum tuberosum sold by Sederma. The agents for stimulating keratinocyte differentiation comprise, for example, minerals such as calcium; the extract of lupin sold by Silab under the trademark Photopreventine®; sodium beta-sitosteryl sulphate sold by Seporga under the trademark Phytocohesine®; and the extract of corn sold by Solabia under the trademark Phytovityl®.

The additional actives may also include other tensioning agents. The term “tensioning agent” in this context means a compound capable of exerting tension on the skin, the effect of which is to temporarily fade out irregularities on the skin's surface, such as wrinkles and fine lines. Among the tensioning agents that may be used in the composition according to the present invention, mention may be made especially of: (1) polyurethane latices or acrylic-silicone latices, in particular those described in patent application EP-1,038,519, such as a propylthio(polymethyl acrylate), propylthio(polymethyl methacrylate) or propylthio(polymethacrylic acid) grafted polydimethylsiloxane, or alternatively a propylthio(polyisobutyl methacrylate) and propylthio (polymethacrylic acid) grafted polydimethylsiloxane. Such grafted silicone polymers are sold especially by 3M under the trademarks VS 80, VS 70 or L021, (2) soybean or wheat plant proteins, and/or (3) sodium magnesium silicates (Laponites). Additional additives may also include those which promote kipolysis. Among the derivatives capable of promoting lipolysis that may be found are: (1) phosphodiesterase inhibitors such as: xanthine derivatives, for instance caffeine and its derivatives, especially the 1-hydroxyalkylxanthines described in document FR-A-2,617,401, caffeine citrate, theophylline and its derivatives, theobromine, acefylline, aminophylline, chloroethyltheophylline, diprofylline, diniprophylline, etamiphylline and its derivatives, etofylline and proxyphylline; combinations containing xanthine derivatives, for instance the combination of caffeine and silanol (caffeine methylsilanetriol derivative), and for example the product sold by Exsymol under the name cafisilane C; compounds of natural origin containing xanthine bases, and especially caffeine, such as extracts of tea, of coffee, of guarana, of mat, of cola (Cola nitida) and especially the dry extract of guarana fruit (Paulina sorbilis) containing 8% to 10% caffeine; ephedrine and its derivatives which may be found especially in natural form in plants such as Ma Huang (Ephedra plant).

Plant extracts and extracts of marine origin which may be used as additional actives include those which are either active on the receptors to be inhibited, such as the beta-2-blockers and the NPY-blockers (described in patent EP-838,217), or inhibit the synthesis of the LDL or VLDL receptors, or are active in stimulating the P receptors and the proteins G, leading to the activation of adenyl cyclase. Examples of plant extracts of this type that may be mentioned include: Garcinia cambogia, extracts of Bupleurum chinensis, extracts of climbing ivy (Hedera helix), of arnica (Arnica montana L), of rosemary (Rosmarinus officinalis N), of marigold (Calendula officinalis), of sage (Salvia officinalis L), of ginseng (Panax ginseng), of St.-John's wort (Hypericum perforatum), of butcher's-broom (Ruscus aculeatus L), of meadowsweet (Filipendula ulmaria L), of orthosiphon (Orthosiphon stamincus Benth), of birch (Betula alba), of pumpwood and of argan tree, extracts of ginkgo biloba, extracts of horsetail, extracts of escin, extracts of cangzhu, extracts of Chrysanthemum indicum, extracts of dioscorea plants rich in diosgenin or pure diosgenin or hecogenin, and derivatives thereof, extracts of plants of the genus Armeniacea, Atractylodis platicodon, Sinom-menum, Pharbitidis or Flemingia, extracts of Coleus such as C. forskohlii, C. blumei, C. esquirolii, C. scutellaroides, C. xanthantus and C. barbatus, such as the extract of the root of Coleus barbatus containing 60% forskolin, extracts of Ballota, extracts of Guioa, of Davallia, of Terminalia, of Barringtonia, of Trema or Antirobia, extracts of marine origin that may be mentioned include: extracts of algae or of phytoplankton, such as rhodysterol or the extract of Laminaria digitata sold under the name PHYCOX75 by Secma, the alga skeletonema described in patent FR-2,782,921 or the diatoms described in patent FR-2,774,292 3) peptides or proteins the peptides derived from parathyroid hormone as described in patents FR-2,788,058 and FR-2,781,231 from Sederma or the peptides described in document FR-A-2,786,693, or alternatively any other peptide with lipolytic properties, protamines and derivatives thereof such as those described in document FR-A-2,758,724.

Active agents acting on the capillary circulation (vasoprotectors or vasodilators) that may be mentioned include flavonoids, ruscogenins, esculosides, escin extracted from common horse-chestnut, nicotinates, heperidine methyl chalcone, ruscus, essential oils of lavender or of rosemary, and extracts of Ammi visnaga. The active agents that are preferred according to the present invention are extracts of corn and of soybean, extracts of the alga Macrocystis pyrifera, polysilicone-8, caffeine, extracts of ivy and Terminalia sericea. They are particularly suitable for compositions intended for redefining the contours of the face.

The amount of lipolytic active agents may vary within a wide range and depends on the nature of the active agent(s) used. In general, the slimming active agent(s) is (are) present in a concentration ranging from 0.001% to 20% and preferably from 0.1% to 10% by weight relative to the total weight of the composition. Indeed, these amounts can be used in connection with any of the possible additional ingredients.

Another cosmetic composition which may be used in accordance with the invention comprise a specific fatty acid-petroselinic acid and/or derivatives thereof, in combination with a Criste Marine or another retinoid-like material derived or extracted form a marine plant, possibly mixed with a retinoid and/or an inhibitor of the enzyme acyl CoA retinol transferase (ARAT) or the enzyme lecithin retinol acyl transferase (LRAT) (hereinafter referred to as LRAT/ARAT inhibitors).

Petroselinic acid (hereinafter referred to as PA) is a monounsaturated long chain (C18) fatty acid which may be used as an additional active.

Derivatives of the free acid which thus comprise petroselinic acid moieties are also contemplated. Preferable derivatives include those derived from substitution of the carboxyl group of the acid, such as esters (e.g. triglyceride esters, monoglyceride esters, diglyceride esters, phosphoesters), amides (e.g. ceramide derivatives), salts (e.g., alkali metal and alkali earth metal salts, ammonium salts); and/or those derived from substitution of the C18 carbon chain, such as alpha-hydroxy and/or beta-hydroxy derivatives.

In the case of triglyceride ester derivatives, all positional isomers of PA substituents on the glycerol backbone are included. The triglycerides must contain at least one PA moiety. For example, of the three esterifiable positions on the glycerol backbone, the 1 and 2 positions may be esterified with PA and by another lipid at position 3 or as an alternative, the glycerol backbone could be esterified by PA at the 1 and 3 positions with another lipid at position 2.

Oils that are rich in petroselinic acid triglyceride are thus also suitable for use in the present invention. Such oils are commercially available and include parsley seed oil, carrot seed oil, fennel fruit oil, parsnip seed oil, coriander seed oil, chervil seed oil, caraway plant oil, and celery seed oil.

The PA to be employed in accordance with the present invention is present in the topical composition in an effective amount. Normally the total amount of the active is present in an amount between 0.0001% and 50% by weight of the composition. More preferably the amount is from 0.01% to 10% and most preferably from 0.1% to 5% in order to maximize benefits at a minimum cost.

Retinoid

The term “retinoid” as used herein in connection with additional moieties may also include, inter alia, retinoic acid, retinoyl ester, retinol, retinyl ester.

The term “retinol” includes the following isomers of retinol: all-trans-retinol, 13-cis-retinol, 11-cis-retinol, 9-cis-retinol, 3,4-didehydro-retinol. Preferred isomers are all-trans-retinol, 13-cis-retinol, 3,4-didehydro-retinol, 9 cis-retinol. Most preferred is all-trans-retinol, due to its wide commercial availability.

Retinyl ester is an ester of retinol. The term “retinol” has been defined above. Retinyl esters suitable for use in the present invention are C₁-C₃₀ esters of retinol, preferably C₂-C₂₀ esters, and most preferably C₂-C₃, and C₁₆ esters because they are more commonly available. The preferred esters for use in the present invention are selected from, retinyl palmitate, retinyl acetate, retinyl propionate and retinyl linoleate, because these are the most commercially available and therefore the cheapest. Retinyl ester is also preferred due to its efficacy.

Retinoyl ester is an ester of retinoic acid. Retinoyl esters suitable for use in the present invention include C₁-C₃₀ esters of retinoic acid, preferably C₂-C₂₀ esters, and most preferably C₂-C₃ and C₁₆ esters. The preferred esters for use in the present invention are selected from retinoyl linoleate, retinoyl palmitate, retinoyl oleate, retinoyl ascorbate, and retinoyl linolenate.

Retinol is an endogenous compound that occurs naturally in the human body and is essential for normal epithelial cell differentiation. Esters of retinol hydrolyze in-vivo to produce retinol. It is believed that retinyl esters and retinol are metabolically converted in the skin into retinoic acid according to the following mechanism

However, most of the endogenously applied retinol is rapidly converted into inactive fatty esters for storage in epidermal cells (keratinocytes).

Esterification of retinol into inactive retinyl esters is achieved in cells by transfer of a fatty acyl group from an acyl CoA, catalyzed by the enzyme acyl CoA retinol transferase (ARAT), or by the transfer of an acyl group from phosphatidyl choline, catalyzed by the enzyme lecithin retinol acyl transferase (LRAT). These esterification reactions are very efficient in keratinocytes—the majority (95%) of cellular retinoids are in the form of retinyl fatty esters.

The term “LRAT/ARAT inhibitor” in the present application thus means an agent which inhibits these esterification reactions and thus potentiates the action of retinol by increasing the amount of retinol available for conversion to retinoic acid. These may also be present as additional actives.

The LRAT/ARAT inhibitors within the scope of the present invention are identifiable as those compounds which at 100 μM concentration inhibit at least 20% of LRAT or ARAT catalyzed retinol esterification as measured by the in vitro Microsomal Assay described below in Example 1. In a preferred embodiment of the invention, the LRAT/ARAT inhibitor is a compound that at 100M concentration inhibits at least 40% and most preferably at least 50% of LRAT or ARAT catalyzed retinol esterification. The in vitro Microsomal Assay employed for determining whether or not a compound is such aLRAT/ARAT inhibitor is as described in Example 1 below.

Thus if a compound passes this in vitro Microsomal assay, that is, it inhibits sufficiently an LRAT or ARAT catalyzed retinol esterification as measured by the in vitro Microsomal Assay, it is included in the present invention even if it is not specifically mentioned herein.

Examples of such LRAT/ARAT inhibitors which satisfy the assay described in Example 1 include fatty acid amides, hydroxy fatty acid amides, ceramides, melinamide, imidazolidinones, and cyclic aliphatic unsaturated hydrocarbons, terpenes, and fatty hydroxyethyl imidazoline surfactants.

Suitable cyclic aliphatic unsaturated compounds are selected according to the in-vitro Microsomal Assay Test described above.

A preferred cyclic aliphatic unsaturated compound is selected from cyclic aliphatic unsaturated aldehydes, ketones, alcohols and esters such as alpha damascone, beta damascone, delta damascone, isodamascone, damascenone, alpha ionone, beta ionone, allyl alpha ionone, isobutyl ionone, alpha methyl ionone, gamma methyl ionone, brahmanol, sandanol, alpha terpineol, lyral, ethyl saffranate, and mixtures thereof. Preferably, in order to maximize performance at a minimum cost, a cyclic aliphatic unsaturated compound is selected from the group consisting of damascones and ionones.

Most preferably, the cyclic aliphatic unsaturated compound is a alpha-Damascone and/or a-Ionone.

Suitable diterpenes are selected according to the in-vitro Microsomal Assay Test described above. A preferred diterpene compound is geranyl geraniol, which is a potent inhibitor of retinol esterification. Fatty hydroxyethyl imidazoline surfactants included in the present invention pass the in-vitro Microsomal Assay test described above. Most preferably, the fatty hydroxyethyl imidazoline is oleyl hydroxyethyl imidazoline, due to its commercial availability and efficacy.

Preferably, the fatty acid amide contains at least 6 carbon atoms. Suitable fatty acids include saturated and unsaturated, straight or branched fatty acids. Suitable fatty acids preferably contain from 8 to 24 carbon atoms, preferably from 12 to 20 carbon atoms, and most preferably from 12 to 18 carbon atoms, because longer chain fatty acid amides are more beneficial for conditioning of the skin. In the most preferred embodiment of the invention, amides of essential fatty acids are employed because essential fatty acids provide nutrition for the skin. Examples of essential fatty acids include but are not limited to linoleic, linolenic, arachidonic, gamma-linolenic, homo-gamma linolenic, and mixtures thereof. Linoleic acid is most preferred because it is also a precursor to ceramide.

The preferred amides included in the present invention are mono-and di-alkanol amides, particularly of essential fatty acids. Alkanol amides are more commonly available than alkyl amides. The most preferred fatty acid amides are selected from mono and diethanolamides and phosphatidylethanolamides of linoleic acid, palmitic acid, and coconut oil; diethyl cocamide, linoleamidyl dimethylamine, dimethyl linoleamide, diethyl linoleamide, dimethyl palmitide, myristoyl sarcosine.

Preferably the hydroxy acid amide is an amide of alpha- or beta-hydroxy acid, i.e., n is 0 or 1. The most preferred hydroxy fatty acid amides to be included in the inventive compositions are: lactamidemonoethanolamide, C₁₃-beta-hydroxy acid amide(2-hydroxy-C₁₃-amide), N-hydroxyethyl-2-hydroxy-C₁₆ amide, 12-hydroxy-N-(2-hydroxyethyl) octadecanamide, and monoethanolamide of castor oil.

A further example of a suitable LRAT/ARAT inhibitor is a PCTA which passes the in vitro Microsomal Assay. Preferably the PTCA is a pentacyclic triterpene monocarboxylic acid. Most preferably, PTCA is selected from the group consisting of ursolic acid, oleanolic acid, glycerrhetinic and glycyrrhizic acid. PTCA is commercially available from Aldrich and Sigma. Plant extracts containing PTCA are suitable for use in the present invention e.g. Rosmarinus officinalis (rosemary), Diospyros spp. (persimmon), Forsythia suspensa (forsythia), Lavandula angustifolia (lavender), Prunella vulgaris (selfheal), Paeonialactifolia, Glycyrrhiza glabra (licorice).

It should be understood that depending on the pH of the composition, PTCA may be present in the composition as a salt, e.g. alkali or alkaline earth salt.

The ceramides may for example be naturally occurring ceramides, phyto ceramides short chain ceramides, pseudoceramides or neoceramides. The general structure of these molecules is described in EP A 711558 whose contents are hereby incorporated by reference.

The most preferred ceramide derivative is acetyl sphingosine due to its efficacy.

The retinoid and/or LRAT/ARAT inhibitor can be included in the inventive compositions in an amount ranging from 0.0001% to 50% by weight of the composition, preferably it is used in an amount of from 0.01% to 10%, most preferably from 0.1% to 5%.

The cosmetic and/or dermatological compositions according to the invention may be in any form that is suitable for topical application, especially in the form of aqueous, aqueous-alcoholic or oily solutions, dispersions of the lotion or serum type, aqueous, anhydrous or oily gels, emulsions of liquid or semi-liquid consistency of the milk type, obtained by dispersing a fatty phase in an aqueous phase (O/W) or conversely (W/O), or a triple emulsion (W/O/W or O/W/o), suspensions or emulsions of soft, semi-solid or solid consistency of the cream or gel type, microemulsions, or alternatively microcapsules, microparticles or vesicular dispersions of ionic and/or nonionic type. These compositions are prepared according to the usual methods using known systems well known to those skilled in the art.

When the composition of the invention is an emulsion, the proportion of the fatty phase may range from 5% to 80% by weight and preferably from 5% to 50% by weight relative to the total weight of the composition. The oils, emulsifiers and co-emulsifiers used in the composition in emulsion form are chosen from those conventionally used in cosmetics or dermatology. The emulsifier, and optionally the co-emulsifier, are present in the composition in a proportion ranging from 0.3% to 30% by weight and preferably from 0.5% to 20% by weight relative to the total weight of the composition.

As oils that may be used in the invention, mention may be made of mineral oils (liquid petroleum jelly), plant oils (liquid fraction of karite butter), animal oils, synthetic oils (purcellin oil or hydrogenated polyisobutene), silicone oils and fluoro oils (perfluoropolyethers). Fatty alcohols, fatty acids and waxes may also be used as fatty substances.

As emulsifiers that may be used in the invention, examples that may be mentioned include fatty acid esters of polyols, such as fatty esters of sorbitol, for instance sorbitan tristearate sold under the name Span 65 by ICI, or fatty esters of glycerol such as glyceryl monostearate, or alternatively PEG esters such as PEG-40 stearate sold under the name Myrj 52 by ICI. They may also be silicone emulsifiers such as the cetyl dimethicone copolyol sold under the name Abil EM90 by Goldschmidt.

In some embodiments, the composition used according to the invention also comprises a dermatologically or cosmetically acceptable vehicle to act as a dilutant, dispersant or carrier for the actives. The vehicle may comprise materials commonly employed in skin care products such as water, liquid or solid emollients, silicone oils, emulsifiers, solvents, humectants, thickeners, powders, propellants and the like.

Among the organic solvents which can act as vehicles are, without limitation, primary alcohols, polyols such as propylene glycol, isoprene glycol, butylene glycol, propylene glycol, glycerol or sorbitol; monoalkyl or dialkyl isosorbide in which the alkyl groups contain from 1 to 5 carbon atoms, for instance dimethyl isosorbide; polyethylene glycols, especially those containing from 6 to 80 ethylene oxides, such as polyethylene glycol 32 OE; ethylene glycol ethers, for instance diethylene glycol monomethyl or monoethyl ether; propylene glycol ethers, for instance dipropylene glycol methyl ether; polyol esters and ethers, such as polypropylene glycol (PPG) esters and more especially fatty acid esters of polypropylene glycol (PPG), fatty alkyl ethers of PPG, for instance PPG-23 oleyl ether and PPG-36 oleate; fatty acid alkyl esters, such as diisopropyl adipate, dioctyl adipate or alkyl benzoates; and mixtures thereof.

Examples of emulsifiers that may be mentioned for the W/O emulsions include dimethicone copolyols, such as the mixture of cyclomethicone and of dimethicone copolyol, sold under the name DC 5225 C by the company Dow Corning or the mixture sold under the name Abil EM 97 by the company Goldschmidt, and alkyldimethicone copolyols, such as the laurylmethicone copolyol sold under the name Dow Corning 5200 Formulation Aid by the company Dow Corning, the cetyldimethicone copolyol sold under the name Abil EM 90 by the company Goldschmidt, or the mixture of cetyldimethicone copolyol, polyglyceryl-4 isostearate and hexyl laurate, sold under the name Abil WE 09 by the company Goldschmidt. One or more co-emulsifiers may also be added thereto, which may be advantageously chosen from the group comprising alkylated esters of polyol. Alkylated esters of polyol that may especially be mentioned include glycerol and/or sorbitan esters, for example polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

Examples of emulsifiers that may be mentioned for the O/W emulsions include nonionic surfactants, and especially esters of polyols and of fatty acids with a saturated or unsaturated chain containing, for example, from 8 to 24 carbon atoms and better still from 12 to 22 carbon atoms, and the oxyalkylenated derivatives thereof, i.e. derivatives containing oxyethylenated and/or oxypropylenated units, such as the glyceryl esters of C₈-C₂₄ fatty acids, and the oxyalkylenated derivatives thereof; the polyethylene glycol esters of C₈-C₂₄ fatty acids, and the oxyalkylenated derivatives thereof; the sorbitol esters of C₈-C₂₄ fatty acids, and the oxyalkylenated derivatives thereof; the sugar (sucrose, glucose or alkylglucose) esters of C₈-C₂₄ fatty acids, and the oxyalkylenated derivatives thereof; fatty alkyl ethers; the sugar ethers of C₈-C₂₄ fatty alcohols, and mixtures thereof.

Glyceryl esters of fatty acids that may especially be mentioned include glyceryl stearate (glyceryl mono-, di- and/or tristearate) (CTFA name: glyceryl stearate) or glyceryl ricinoleate, and mixtures thereof.

Polyethylene glycol esters of fatty acids that may especially be mentioned include polyethylene glycol stearate (polyethylene glycol mono-, di- and/or tristearate) and more especially polyethylene glycol 50 OE monostearate (CTFA name: PEG-50 stearate) and polyethylene glycol 100 OE monostearate (CTFA name: PEG-100 stearate), and mixtures thereof.

Mixtures of these surfactants may also be used, for instance the product containing glyceryl stearate and PEG-100 stearate, sold under the name Arlacel 165 by the company Uniqema, and the product containing glyceryl stearate (glyceryl mono-distearate) and potassium stearate, sold under the name Tegin by the company Goldschmidt (CTFA name: glyceryl stearate SE).

Fatty acid esters of glucose or of alkylglucose that may be mentioned in particular include glucose palmitate, alkylglucose sesquistearates, for instance methylglucose sesquistearate, alkylglucose palmitates, for instance methylglucose palmitate or ethylglucose palmitate, fatty esters of methylglucoside and more especially the diester of methylglucoside and of oleic acid (CTFA name: Methyl glucose dioleate); the mixed ester of methylglucoside and of the oleic acid/hydroxystearic acid mixture (CTFA name: Methyl glucose dioleate/hydroxysterate); the ester of methylglucoside and of isostearic acid (CTFA name: Methyl glucose isostearate); the ester of methylglucoside and of lauric acid (CTFA name: Methyl glucose laurate); the mixture of the monoester and diester of methylglucoside and of isostearic acid (CTFA name: Methyl glucose sesquiisostearate); the mixture of the monoester and diester of methylglucoside and of stearic acid (CTFA name: Methyl glucose sesquistearate) and in particular the product sold under the name Glucate SS by the company Amerchol, and mixtures thereof.

Examples of oxyethylenated ethers of a fatty acid and of glucose or of alkylglucose that may be mentioned include the oxyethylenated ethers of a fatty acid and of methylglucose, and in particular the polyethylene glycol ether of the diester of methyl glucose and of stearic acid containing about 20 mol of ethylene oxide (CTFA name: PEG-20 methyl glucose distearate), such as the product sold under the name Glucam E-20 distearate by the company Amerchol; the polyethylene glycol ether of the mixture of monoester and diester of methylglucose and of stearic acid containing about 20 mol of ethylene oxide (CTFA name: PEG-20 methyl glucose sesquistearate) and in particular the product sold under the name Glucamate SSE-20 by the company Amerchol, and the product sold under the name Grillocose PSE-20 by the company Goldschmidt, and mixtures thereof.

Examples of sucrose esters that may be mentioned include sucrose palmitostearate, sucrose stearate and sucrose monolaurate.

Examples of fatty alkyl ethers that may be mentioned include polyethylene glycol ethers of fatty alcohols containing from 8 to 30 carbon atoms and especially from 10 to 22 carbon atoms, such as polyethylene glycol ethers of cetyl alcohol, of stearyl alcohol or of cetearyl alcohol (mixture of cetyl alcohol and stearyl alcohol). Examples that may be mentioned include ethers comprising from 1 to 200 and preferably from 2 to 100 oxyethylene groups, such as those of CTFA name Ceteareth-20 and Ceteareth-30, and mixtures thereof.

Sugar ethers that may especially be mentioned are alkylpolyglucosides, for example decylglucoside, for instance the product sold under the name Mydol 10 by the company Kao Chemicals, the product sold under the name Plantaren 2000 by the company Cognis, and the product sold under the name Oramix NS 10 by the company SEPPIC; caprylyl/capryl glucoside, for instance the product sold under the name Oramix CG 110 by the company SEPPIC or under the name Lutensol GD 70 by the company BASF; laurylglucoside, for instance the products sold under the names Plantaren 1200 N and Plantacare 1200 by the company Cognis; cocoglucoside, for instance the product sold under the name Plantacare 818/UP by the company Cognis; cetostearyl glucoside optionally as a mixture with cetostearyl alcohol, sold, for example, under the name Montanov 68 by the company SEPPIC, under the name Tego-Care CG90 by the company Goldschmidt and under the name Emulgade KE3302 by the company Cognis; arachidyl glucoside, for example in the form of the mixture of arachidyl alcohol and behenyl alcohol and arachidyl glucoside, sold under the name Montanov 202 by the company SEPPIC; cocoylethylglucoside, for example in the form of the mixture (35/65) with cetyl alcohol and stearyl alcohol, sold under the name Montanov 82 by the company Seppic; and mixtures thereof.

When the composition is in the form of an emulsion, the nature of the oily phase of the emulsion is not critical. The oily phase may thus consist of any fatty substance and especially any oil conventionally used in cosmetics or dermatology. The oily phase generally comprises at least one oil, especially a cosmetic oil.

As oils that may be used in the composition of the invention, mention may be made for example of:

hydrocarbon-based oils of animal origin, such as perhydrosqualene;

hydrocarbon-based oils of plant origin, such as liquid triglycerides of fatty acids containing from 4 to 10 carbon atoms, for instance heptanoic or octanoic acid triglycerides or alternatively, for example, sunflower oil, corn oil, soybean oil, marrow oil, grapeseed oil, sesame oil, hazelnut oil, apricot oil, macadamia oil, coriander oil, arara oil, sunflower oil, castor oil, avocado oil, caprylic/capric acid triglycerides, for instance those sold by the company Stearineries Dubois or those sold under the names Miglyol 810, 812 and 818 by the company Dynamit Nobel, jojoba oil or shea butter oil;

substantially linear or branched hydrocarbons of mineral or synthetic origin, such as volatile or non-volatile liquid paraffins, and derivatives thereof, petroleum jelly, polydecenes, isohexadecane, isododecane and hydrogenated polyisobutene such as Parleam® oil;

fatty alcohols containing from 8 to 26 carbon atoms, for instance cetyl alcohol, stearyl alcohol and the mixture thereof (cetylstearyl alcohol), octyldodecanol, 2-butyloctanol, 2-hexyldecanol, 2-undecylpentadecanol, oleyl alcohol or linoleyl alcohol;

alkoxylated fatty alcohols and especially ethoxylated fatty alcohols, such as oleth-12, ceteareth-12 and ceteareth-20;

partially hydrocarbon-based and/or partially silicone-based fluoro oils, for instance those described in document JP-A-2 295 912. Examples of fluoro oils that may also be mentioned include perfluoromethylcyclopentane and perfluoro-1,3-dimethylcyclohexane, sold under the names “Flutec PC1®” and “Flutec PC®” by the company BNFL Fluorochemicals; perfluoro-1,2-dimethylcyclobutane; perfluoroalkanes such as dodecafluoropentane and tetradecafluorohexane, sold under the names “PF 5050®” and “PF 5060®” by the company 3M, or alternatively bromoperfluorooctyl sold under the name “Foralkyl®” by the company Atochem; nonafluoromethoxybutane sold under the name “MSX 4518®” by the company 3M and nonafluoroethoxyisobutane; perfluoromorpholine derivatives, such as the 4-trifluoromethylperfluoromorpholine sold under the name “PF 5052®” by the company 3M;

silicone oils, for instance volatile or non-volatile polymethylsiloxanes (PDMSs) containing a substantially linear or cyclic silicone chain, that are liquid or pasty at room temperature, especially cyclopolydimethylsiloxanes (cyclomethicones) such as cyclohexadimethylsiloxane and cyclopentadimethylsiloxane; polydimethylsiloxanes comprising alkyl, alkoxy or phenyl groups, that are pendant or at the end of a silicone chain, these groups containing from 2 to 24 carbon atoms; phenylsilicones, for instance phenyl-trimethicones, phenyldimethicones, phenyltrimethylsiloxydiphenylsiloxanes, diphenyldimethicones, diphenylmethyldiphenyltrisiloxanes, 2-phenylethyl-trimethysiloxysilicates and polymethylphenylsiloxanes;

mixtures thereof.

In the list of oils mentioned above, the expression “hydrocarbon-based oil” means any oil mainly comprising carbon and hydrogen atoms, and optionally ester, ether, fluoro, carboxylic acid and/or alcohol groups.

The other fatty substances that may be present in the oily phase are, for example, fatty acids containing from 8 to 30 carbon atoms, for instance stearic acid, lauric acid, palmitic acid and oleic acid; waxes, for instance lanolin, beeswax, carnauba wax or candelilla wax, paraffin waxes, lignite wax or microcrystalline waxes, ceresin or ozokerite, synthetic waxes such as polyethylene waxes, Fischer-Tropsch waxes; liquid petroleum paste; gums such as silicone gums (dimethiconol); silicone resins such as trifluoromethyl-C1-4-alkyldimethicone and trifluoropropyldimethicone.

These fatty substances may be chosen in a varied manner by a person skilled in the art so as to prepare a composition having the desired properties, for example in terms of consistency or texture.

The vehicle will usually form from 5% to 99.9%, preferably from 25% to 80% by weight of the composition, and can, in the absence of other cosmetic adjuncts, form the balance of the composition.

In one particular embodiment, the compositions of the present invention are in the form of a cream or firming serum.

In a known manner, the cosmetic and/or dermatological compositions of the invention may also contain adjuvants that are common in the fields under consideration, such as, without limitation, hydrophilic or lipophilic gelling agents, hydrophilic or lipophilic agents, preserving agents, antioxidants, solvents, fragrances, organic fillers, inorganic fillers, screening agents, film formers, antioxidants, sequestering agents, pigments, odor absorbers and dyestuffs. For a list of individual components falling within these categories, see for example the International Cosmetic Ingredient Dictionary and Handbook, 9th ed., 2002 (Pepe, Wenninger & McEwen, Jr. eds.), published by the Cosmetic, Toiletry and Fragrance Associate, 1101 17th Street, NW, Ste. 300, Washington D.C. 20036-4702, the text of which is hereby incorporated by reference. The amounts of these various adjuvants are those conventionally used in the field under consideration, and, for example, from 0.01% to 20% each relative to the total weight of the composition. Depending on their nature, these adjuvants may be introduced into the fatty phase if any, into the aqueous phase if any, into the lipid vesicles if any, and/or into the nanoparticles if any. In any case, these adjuvants and the proportions thereof will be chosen so as not to harm the properties desired according to the invention.

As hydrophilic gelling agents other than the polymers described above, examples that may be mentioned include carboxyvinyl polymers such as the Carbopol products (carbomers) and the Pemulen products (acrylate/C₁₀-C₃₀-alkylacrylate copolymer); polyacrylamides, for instance the crosslinked copolymers sold under the name Sepigel 305 (CTFA name: polyacrylamide/C₁₃-₁₄ isoparaffin/Laureth-7) or Simulgel 600 (CTFA name: acrylamide/sodium acryloyldimethyltaurate copolymer/isohexadecane/polysorbate 80) by the company SEPPIC; cellulose derivatives such as hydroxyethylcellulose; polysaccharides and especially gums such as xanthan gum; and mixtures thereof.

Besides the actives, vehicles and other adjuvants, other specific skin-benefit actives such as sunscreens, other skin lightening agents, skin tanning agents may also be included. The vehicle may also further include adjuncts such as perfumes, opacifiers, preservatives, colorants and buffers.

To prepare the topical composition used in the method of the present invention, the usual manner for preparing skin care products may be employed. The active components are generally incorporated in a dermatologically/cosmetically acceptable carrier in conventional manner. The active components can suitably first be dissolved or dispersed in a portion of the water or another solvent or liquid to be incorporated in the composition. The preferred compositions are oil-in-water or water-in-oil or water-in-oil-in-water or oil-in-water-in-oil emulsions.

The composition may be packaged in any suitable manner such as in a jar, a bottle, tube, roll-ball, or the like, in the conventional manner. It is also envisaged that the inventive compositions could be packaged as a kit.

In addition, the compositions of the invention will be used for a time that is sufficient to obtain the expected effects according to the invention. These compositions can be applied one or more times a day. To give an order of magnitude, this duration may be at least 15 days, but it may also be more than 4 weeks or even more than 8 weeks. The minimum amount applied is generally about 0.5 micrograms per cm² of skin surface.

### EXAMPLE I

### Cryste Marine (0.75%) Serum

| Phase | Ingredient | % w/w | 1200 |
|---|---|---|---|
| A | DI Water | q.s. | q.s. |
| | Preservative | 0.4000 | 8.000 |
| | Glycerin | 5.0000 | 100.000 |
| | Butylene Glycol | 2.0000 | 40.000 |
| | DIMETHICONE COPOLYOL | 2.0000 | 40.000 |
| B | AMPS | 1.0000 | 20.000 |
| C | HYDROLYZED RICE PROTEIN | 1.5000 | 30.000 |
| | SOYBEAN PROTEIN | 0.1000 | 2.000 |
| | AQUA (and) FAGUS SYLVATICA | 2.0000 | 40.000 |
| | COLLAGEN (and) CHITOSAN (and) | 3.0000 | 60.000 |
| | SODIUM CHONDROITIN SULFATE | | |
| | criste marine complex (5%) | 1.5000 | 30.000 |
| | Vit. E Acetate | 0.1000 | 2.000 |
| | Padena Pavonica (10%) | 0.7500 | 15.000 |
| | Cyclomethicone | 7.0000 | 140.000 |
| | Polysorbate 20 | 0.1000 | 2.000 |
| | Alcohol | 5.0000 | 100.000 |
| | TOTAL | 100.0000 | 100.000 |

Procedure: 1 L Greerco Mixer.

Phase A: Add water to main breaker and heat to 40 C. Pre-mix the preservative in butylene glycol and add to main breaker. Add rest of phase A ingredients one by one with mixing Heat Phases A to 80 C and mix until all is dissolved.

Phase B: Sprinkle into main beaker and mix well. Make sure polymer is completely hydrated. Mix for 10 minutes. Maintain temperature. Begin cooling to 40-45 C.

Phase C: Add to main breaker one by one with mixing in between. Mix well.

### EXAMPLE II

### Cryste Marine (0.025%) Eye Cream

| | | EXT. FACT: 25 | |
|---|---|---|---|
| Phase | INCI name | % W/W | EXT. WT. |
| A | WATER | 71.64 | 1791.00 |
| | ACRYLATES/C10-30 ALKYL ACRYLATE | 0.40 | 10.00 |
| | CROSSPOLYMER | | |
| | Glycerin | 7.00 | 175.00 |
| | Preservative | 0.50 | 12.50 |
| B | POLY C10-30 ALKYL ACRYLATE | 2.00 | 50.00 |
| | STEARIC ACID | 1.50 | 37.50 |
| | BEESWAX | 1.00 | 25.00 |
| | PROPYLPARABEN | 0.10 | 2.50 |
| | GLYCERYL STEARATE SE | 0.80 | 20.00 |
| | SQUALANE | 0.00 | 0.00 |
| | CYCLOHEXASILOXANE | 0.00 | 0.00 |
| | SUCROSE STEARATE | 0.00 | 0.00 |
| | BUTYROSPERMUM PARKII (SHEA BUTTER) | 4.00 | 100.00 |
| | CAPRYLIC/CAPRIC TRIGLYCERIDE | 0.50 | 12.50 |
| | (and) CRITHMUM MARITIMUM EXTRACT (5%) | | |
| | Vit E acetate | 0.01 | 0.25 |
| C | WATER | 2.00 | 50.00 |
| | SODIUM HYDROXIDE | 0.05 | 1.25 |
| D | CYCLOPENTASILOXANE (and) DIMETHICONOL | 1.00 | 25.00 |
| | CYCLOPENTASILOXANE | 3.50 | 87.50 |
| | ALUMINUM STARCH OCTENYLSUCCINATE | 2.50 | 62.50 |
| E | COLLAGEN (and) CHITOSAN (and) SODIUM | 1.250 | 31.25 |
| | CHONDROITIN SALFATE | | |
| | PARAFFIN (and) PADINA PAVONICA EXTRACT | 0.250 | 6.25 |
| | (10%) | | |
| TOTAL | | 100.00 | 2500.00 |

Procedure: EL-1 ESCO Processor

Phase A: Introduce water into the main kettle, slowly sprinkle the polymer. Add the preservative and then heat to 80 C and mix till clear. Once uniform cool to 25 C.

Phase B: Heat Phase B to 80 C.

Emulsify: Add B into A and homogenize 10 minutes. Have nice emulsion after that. Cool to 60° C.

Phase C: Dissolve sodium hydroxide into water. Add phase C to the main kettle and continue mixing till homogenous.

Phase D: Add-all ingredients in that phase and mix until homogeneous. Add phase D to the main kettle and continue mixing till homogenous.

Phase E: Add the ingredients in phase A separately to the batch and mix until homogeneous.

### EXAMPLE 3

### Criste Marine (0.05%) Cream

OBJECTIVE:

| PHASE | INCI name | % W/W |
|---|---|---|
| A | WATER | 71.8500 |
| | GLYCERIN | 5.000 |
| | PROPYLENE GLYCOL | 1.000 |
| | SODIUM HYDROXIDE | 0.030 |
| | Preservative | 0.300 |
| | PANTHENOL | 0.500 |
| B | PEG-20 STEARATE | 0.800 |
| | GLYCERYL STEARATE (and) PEG-100 | 2.000 |
| | STEARATE | |
| | CETYL ALCOHOL | 1.500 |
| | STEARYL ALCOHOL | 1.500 |
| | STEARIC ACID | 3.000 |
| | BEESWAX | 1.000 |
| | HYDROXYPALMITOYL SPHINGANINE | 0.020 |
| | SQUALANE | 4.000 |
| C | POLYACRYLAMIDE (and) C13-14 | 0.500 |
| | ISOPARAFFIN (and) LAURETH-7 | |
| D | SOYBEAN PROTEIN | 0.500 |
| | PARAFFIN (and) PADINA PAVONICA | 0.500 |
| | EXTRACT (10%) | |
| | ALUMINUM STARCH OCTENYLSUCCINATE | 2.500 |
| | CAPRYLIC/CAPRIC TRIGLYCERIDE | 1.000 |
| | (and) CRITHMUM MARITIMUM (5%) | |
| | COLLAGEN (and) CHITOSAN (and) | 2.500 |
| | SODIUM CHONDROITIN SULFATE | |
| TOTAL | | 100.000 |

1. Under moderate speed homogenization Heat A (main) to 80 CC.

2. Heat B (oil phase) to 80 C.

3. Emulsify: Add phase B to phase and continuing with moderate speed homogenization. Homomix for 10 minutes then start cooling down.

4. Add phase C at 60-65 C and mix for 10 minutes (until smooth).

5. Add phase all ingredients of phased D separately at 45 C. Mix the batch between additions until uniform

6. Cool to 30 C under sweep—STOP.

### EXAMPLE 4

Tests were run to evaluate the eye cream formulation of Example II for skin firmness. This formulation was found to statistically significantly increase the firmness, resilience and extensibility of skin to which it was applied four (4) hours after treatment compared to the untreated control. Twenty seven panelists, three male and twenty-four female were tested to determine the biomechanical properties (i.e. extensibility, resilience, and firmness or elasticity) of the skin after product application. Although twenty-seven (27) panelists were recruited, one (1) panelist missed her four (4) hour time point. Therefore, the sample size was twenty-six (26) at four (4) hours. The test was run generally using the following protocol:

Materials

Dermal Torque Meter (Dia-Stron)

Alcohol wipes or tissues

Micropipettors

9 Finger cots or glass rods

Microbalance

Test product(s)

Testing Conditions

Temperature: Average Temp. 71.8° F. and 24% relative humidity.

Procedure

Conduct the study in a double-blind fashion.

Record the temperature and the relative humidity.

Ensure that no residual product(s) is present on the sites to be tested. Provide specific instructions to the subjects to not use any topical products on their volar forearms for three (3) days prior to the test date. Then, gently wipe the entire volar forearms of each subject with a soft tissue. In the event that it cannot be ascertained whether the subjects followed the instructions, cleanse each subject's volar forearms with a sterile alcohol preparatory pad (seventy percent [70%) isopropyl alcohol). Allow the alcohol to dry for ten (10) minutes.

Mark 4-cm×4-cm areas on the volar forearms of each panelist for each test product and its corresponding control site.

Follow the randomization schedule on all test sites and take initial measurements (i.e., extensibility [Ue], resilience [Ur], firmness [Ur/Ue]) on both arms at baseline (T0), using the Dermal Torque Meter (Dia Stron).

Note: If the first measurement produced an abnormal curve, a second reading should be taken in an area close to the site of the first measurement. No more than two (2) measurements should be taken per site. When two (2) measurements are taken and both produce abnormal curves, both measurements should be excluded from the analysis.]

Following the randomization schedule, apply an amount of product equal to ˜2 mg/cm² to each test site(s). For liquid-type products (i.e., most lotions and creams), use a micropipettor to draw thirty-two (32) μl of the test product. If the product is too thick to be drawn by a micropipettor, weigh the appropriate amount on a microbalance scale, and use a glass rod or a finger cot to spread the product. The untreated site(s) serves as the control(s).

Take biomechanical property measurements four (4) hours (or other specified time[s]) after product application.

Record all readings in the raw data and provide an explanation of all data that will be excluded from analysis.

Preserve all readings in the sorted data and preserve all the explanations.

Analysis of Results

To compare the baseline values of Ue, Ur, and Ur/Ue of the untreated and the treated sites, use a paired-t test.

Compare the change in the Ue, Ur, and Ur/Ue values from baseline to four (4) hours (or other specified time) using a paired t-test.

If the data are not normally distributed, use a nonparametric statistical test, the Wilcoxon Signed Rank Test (see GIP-004).

In the case of the material produced as described in Example II, a paired “t” test was used to compare the baseline values of Ue, Ur, and Ur/Ue of the untreated sites and the treated sites. The change in the Ur/Ue, Ur, and Ue values from baseline to four (4) hours (T4 hours-Baseline) of the untreated sites and the treated sites was compared using a paired “t” test. The statistical significance level was set at p-Valve ≦0.05.

Results:

TABLE 1
Comparison of Ur/Ue Values for Firmness Untreated
(Control) Site vs. Treated Eye Cream Sites on the
Volar Inner Forearm Mean (Standard Deviation)

| | | Untreated Site | | |
|---|---|---|---|---|
| | Sample | (Control) | Treated Site | |
| Time Period | Size | [Site B] | [Site A] | P-Value |
| Baseline | 27 | 0.39 | 0.39 | 0.774 |
| | | (±0.04) | (±0.06) | |
| Diff-Four (4) Hours | 26 | −0.01 | 0.02 | 0.007 |
| After Application | | (±0.04) | (±0.05) | |

There were no statistically significant differences in firmness between the control sites and the treated sites at baseline. There was a statistically significant increase in firmness for the treated sites compared to the untreated control sites four (4) hours after treatment.

TABLE 2
Comparison of Ur Values for Resilience Untreated
(Control) Site vs. Treated Eye Cream Sites on the
Volar Inner Forearm Mean (Standard Deviation)

| | | Untreated Site | | |
|---|---|---|---|---|
| | Sample | (Control) | Treated Site | |
| Time Period | Size | [Site B] | [Site A] | P-Value |
| Baseline | 27 | 0.73 | 0.71 | 0.307 |
| | | (±0.17) | (±0.16) | |
| Diff-Four (4) Hours | 26 | 0.02 | 0.18 | <0.001 |
| After Application | | (±0.09) | (±0.13) | |

There were no statistically significant differences in resilience between the control sites and the treated sites at baseline. There was a statistically significant increase in resilience for the treated sites compared to the untreated control sites four (4) hours after treatment.

TABLE 3
Comparison of Ur Values for Extensibility Untreated
(Control) Site vs. Eye Cream Sites on the Volar
Inner Forearm Mean (Standard Deviation)

| | | Untreated Site | | |
|---|---|---|---|---|
| | Sample | (Control) | Treated Site | |
| Time Period | Size | [Site B] | [Site A] | P-Value |
| Baseline | 27 | 1.91 | 1.85 | 0.227 |
| | | (±0.37) | (±0.37) | |
| Diff-Four (4) Hours | 26 | 0.13 | 0.38 | <0.001 |
| After Application | | (±0.23) | (±0.23) | |

There were no statistically significant differences in resilience between the control sites and the treated sites at baseline. There was a statistically significant increase in extensibility for the treated sites compared to the untreated control sites four (4) hours after treatment.

### CONCLUSION

According to this study, the Eye Cream formulation of Example II was found to statistically significantly increase the firmness, resilience and extensibility of the skin four (4) hours after treatment, compared to the untreated control.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cosmetic or dermatological topical composition comprising an extract from Criste Marine in an amount of at least about 0.025% by weight of said composition and an extract from Padina Pavonica in an amount of at least about 0.025% by weight of said composition and at least one dermatologically or cosmetically acceptable vehicle.

2. The composition of claim 1 further comprising ascorbic acid or a derivative or analog thereof, provided in an amount of between about 0.001 and about 20.0% by weight of said composition.

3. The composition as in any one of claims 1 or 2, wherein the amount of said extract of Criste Marine ranges from between about 0.025% and about 10.0%.

4. The composition of claim 3, wherein the amount of said extract of Criste Marine ranges from between about 0.025% and about 5.0%.

5. The composition as in any one of claims 1 or 2, wherein the amount of said extract of Padina Pavonica ranges from between about 0.025% and about 10.0%.

6. The composition of claim 5, wherein the amount of said extract of Padina Pavonica ranges from between about 0.025% and about 5.0%.

7. A cosmetic or dermatological topical composition consisting essentially of an extract from Criste Marine and an extract from Padina Pavonica in amounts which are effective to provide such compositions with an improvement in firmness orelasticity of skin to which it is applied or a softening in the look of lines or wrinkles thereof.

8. The composition of claim 7 further comprising ascorbic acid or a derivative or analog thereof, provided in an amount of between about 0.001% and about 20.0%.

9. The composition as in any one of claims 7 or 8, wherein the amount of said extract of Criste Marine ranges from between about 0.025% and about 10%.

10. The composition of claim 9, wherein the amount of said extract of Criste Marine ranges from between about 0.025% and about 5.0%.

11. The composition as in any one of claims 7 or 8, wherein the amount of said extract of Padina Pavonica ranges from between about 0.025% and about 10.0%.

12. The composition of claim 11, wherein the amount of said extract of Padina Pavonica ranges from between about 0.025% and about 5.0%.

13. The composition of claim 1 being in the form of a cream, lotion, ointment, gel, emulsion or serum.

14. The composition as in any one of claims 1 or 2, further comprising at least one adjuvant.

15. The composition as in any one of claims 1 or 2, wherein said Padina Pavonica extract exhibits a biological activity of at least about 100,000 U Tex/Liter.

16. The composition as in any one of claims 1 or 2, wherein said Criste Marine extract has at least about 350 mg/kg of phytosterols or at least about 100 mg/kg tocopherols.

17. The composition as in any one of claims 1 or 2, wherein said Criste Marine extract has at least about 350 mg/kg of phytosterols and at least about 100 mg/kg tocopherols.

18. A method of improving the firmness or elasticity of skin or visibly softening lines or wrinkles of skin comprising the steps of applying to skin in need thereof of a composition that comprises extracts from Criste Marine and an extract from Padina Pavonica, at least once a day and repeating the application for a time sufficient to provide either improved firmness, improved elasticity or visibly softened look to lines and wrinkles of the skin to which it is applied.
